(19) United States
(12) Patent Application Publication
(10) Pub. No.: US 2026/0151255 A1
(43) Pub. Date: Jun. 4, 2026

(54) DENTAL SPLINT

(71) Applicant: ALWAFLEX AG, Wettingen (CH)

(72) Inventor: Egon ALRAUN, Lengnau (CH)

(21) Appl. No.: 19/398,255

(22) Filed: Nov. 24, 2025

(30) Foreign Application Priority Data
Nov. 30, 2024 (EP) 24216700.5

(51) Int. Cl. A61F 5/56

(52) U.S. Cl. CPC A61F 5/566; A61F 2005/563

(57) ABSTRACT

A dental splint for locking the lower jaw, preferably in a predefined protrusion relationship of upper and lower jaw, including an upper jaw shell, a lower jaw shell and a connecting bar. The upper and lower jaw shells are fixedly connected to each other via the connecting bar. The dental splint is configured so that saliva ducts are present between the dental splint and the chewing surface in the region of the chewing surfaces and ensures permanent flushing of saliva under the dental splint while the dental splint is being worn during sleep.

### CROSS-REFERENCE TO RELATED APPLICATION

The present application claims priority of EP 24216700.5, filed Nov. 30, 2024, the priority of this application is hereby claimed, and this application is incorporated herein by reference.

### BACKGROUND OF THE INVENTION

The invention relates to a dental splint for locking the lower jaw, preferably in a predefined protrusion ratio of the upper and lower jaw, comprising an upper jaw shell, a lower jaw shell and a connecting bar, wherein the upper and lower jaw shells are fixedly connected to one another via the connecting bar.

Dental splints are usually used to treat snoring, sleep apnea, tongue biting, cheek biting and teeth grinding during the night. For this purpose, the lower jaw is brought into a protrusive and open parking position during the night and held in this position, which is known from the prior art as a lower jaw protrusion splint. Most of the known splints consist of an upper and lower jaw splint, each of which is designed separately and connected to each other with the aid of a hinge or non-fixed connecting element. The production is correspondingly complex and the wearing comfort leaves much to be desired.

US 2014/0076333 discloses a dental splint for the treatment of sleep apnea, wherein the splint is formed from one part and there is no separate splint for the upper and lower jaw. The position of the teeth of the lower and upper jaw in relation to each other is precisely defined, with the dental splint resting directly against the teeth everywhere.

EP 1 139 905 A1 discloses a mouthguard mainly for sports applications. The mouthguard has a stable outer U-shaped shell in which an inner shell adapted to the teeth is arranged. Such a splint is not suitable for wearing to treat the aforementioned sleep problems, rather such a splint serves purely to protect the teeth, mainly during sporting activities.

The disadvantage of the above-mentioned dental splints is that pools of saliva form under the splints in the fissures of the chewing surfaces and in the interdental spaces and cannot drain away. This can result in an excessive build-up of bacteria in these areas. This can result in accelerated tooth decay and gum disease.

### SUMMARY OF THE INVENTION

It is the object of the invention to propose a dental splint for the treatment of snoring, sleep apnea, tongue biting, cheek biting and teeth grinding during the night, which ensures a high level of wearing comfort during sleep and the formation of dental caries and gum disease or deposits can be avoided by preventing the accumulation of pools of saliva.

According to the invention, this object is achieved in that the dental splint is designed in such a way that saliva ducts are present between the dental splint and the chewing surface of the teeth in the region of the chewing surfaces and ensure permanent saliva flushing under the dental splint while the dental splint is being worn during sleep.

The dental splint according to the invention serves to lock the lower jaw, preferably in a predefined protrusion ratio of the upper and lower jaw. The protrusive position creates more space in the throat and at the same time trains the throat muscles during sleep via the rows of teeth of the upper and lower jaw, which promotes the treatment of snoring, sleep apnea, tongue biting, cheek biting and teeth grinding.

The dental splint according to the invention comprises an upper jaw shell, a lower jaw shell and a connecting bar, wherein the upper and lower jaw shells are connected to one another via the connecting bar. The upper and lower jaw shells are fixedly or immovably connected to each other via the connecting bar. The dental splint preferably has a flexible material, whereby the entire dental splint is flexible. The dental splint according to the invention is designed in such a way that saliva ducts are present between the dental splint and the chewing surface in the region of the chewing surfaces, which ensure permanent saliva flushing under the dental splint while the dental splint is being worn during sleep.

This means that the dental splint does not rest against the chewing surfaces of the teeth in the region of the chewing surfaces, but instead cavities are formed there between the tooth or the chewing surface and the dental splint, which are formed as saliva ducts.

Preferably, the saliva ducts are formed by the fact that the dental splint has no contact with the teeth in the area of the fissures of the occlusal chewing surfaces, which results in saliva ducts that can be flushed under between the chewing surfaces and the dental splint. This allows saliva to flow freely around the teeth at all times and prevents bacteria from forming under the inserted sleep splint.

Preferably, the saliva ducts are formed between a saliva duct edge on the dental splint and the occlusal chewing surfaces of the teeth. The saliva duct edge arranged on the dental splint is directed towards the chewing surface, but is not in contact with it, whereby the saliva ducts are formed under the dental splint. The saliva ducts are preferably located in the molar, premolar and palatal regions of the upper and lower jaw.

It has been shown to be advantageous if the saliva ducts run along the fissures of the chewing surfaces. The fissures and the offset saliva duct edge form the saliva duct under the dental splint, which ensures the flushing of the teeth and saliva flow.

It has been shown to be a preferred design if the upper jaw shell, the lower jaw shell and the connecting bar are formed together as one part. This relieves the temporomandibular joints during sleep and keeps them in this position so that they cannot shift out of the protruded advancement position.

Preferably, the dental splint has interdental openings, wherein the interdental openings are positioned between the teeth. The interdental openings give the dental splint greater flexibility. The interdental openings are preferably arranged in the buccal, labial, palatal and/or lingual region.

It has been shown to be advantageous if the interdental openings and the saliva ducts arranged in the region of the chewing surfaces are connected to each other via interdental passages. This enables the teeth to be flushed and ensures that the saliva can flow out.

It has been shown to be advantageous if the dental splint has buccally arranged openings.

Preferably, the dental splint has labial openings.

The buccal and labial openings are preferably aligned with the tooth surface. Preferably, they are located above the widest point of the respective tooth in the lower jaw shell and below the widest point of the respective tooth in the upper jaw shell. The arrangement of the buccal and labial openings at the intended locations ensures that the dental splint is well fixed to the teeth, as the splint is given increased flexibility and elasticity by the openings, which allows the dental splint to adapt to the shape of the tooth. In addition, the buccal and labial openings also support the circulation of saliva on the tooth surface.

It is advantageous if the dental splint is designed in such a way that the tooth necks are exposed. The free circulation of saliva is also ensured by the fact that all tooth necks are exposed around the splint and are not touched by the splint. It is advantageous if the tooth necks in the upper and lower jaw are exposed palatally, buccally, lingually and labially.

A preferred embodiment has been shown to be when a breathing opening is located in the labial area between the upper and lower jaw shell. This enables good and easy breathing during sleep.

Preferably, the dental splint is designed as a 3D printed part. This ensures individual adaptation and adjustment of the tooth contours to a dental splint.

It is advantageous if the dental splint is made of a nylon material, preferably a medically certified biocompatible nylon material. This material ensures a medically safe application as well as that the surfaces are smooth, which ensures a high wearing comfort and that this material is hardly breakable. It is advantageous if the surface of the dental splint is chemically vapor-smoothed, which seals the splint and prevents bacteria from penetrating the material.

All design options can be freely combined with one another.

The various features of novelty which characterize the invention are pointed out with particularity in the claims annexed to and forming a part of the disclosure. For a better understanding of the invention, its operating advantages, specific objects attained by its use, reference should be had to the drawings and descriptive matter in which there are illustrated and described preferred embodiments of the invention.

### BRIEF DESCRIPTION OF THE DRAWING

FIG. 1 a three-dimensional view of a dental splint according to the invention arranged on a set of teeth;

FIG. 2 a three-dimensional view of a dental splint according to the invention;

FIG. 3 the course of the saliva ducts in the lower jaw; and

FIG. 4 a cross-section of the dental splint at one point of the jaw.

### DETAILED DESCRIPTION OF THE INVENTION

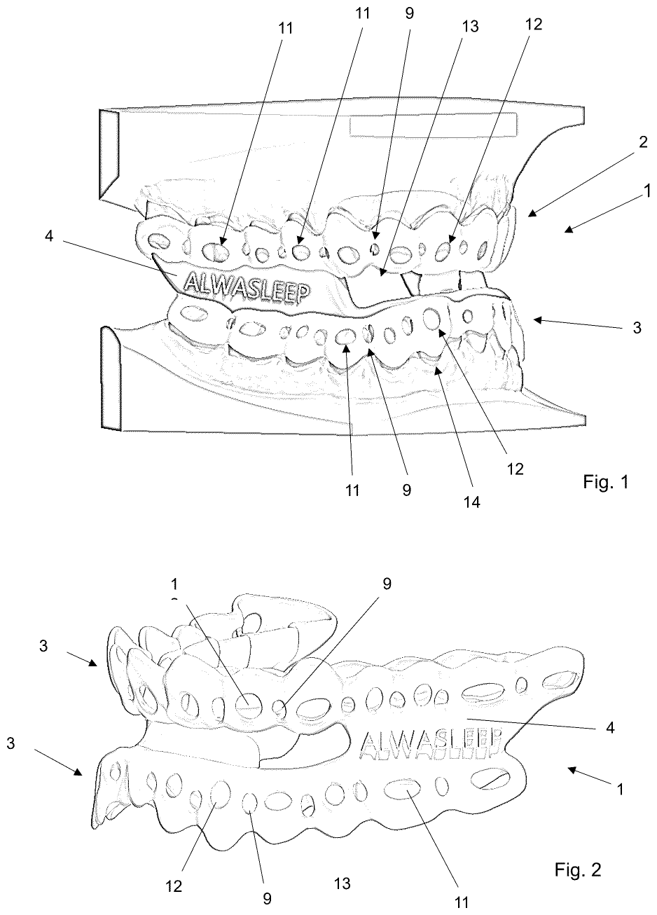

The drawing shown in FIG. 1 shows a three-dimensional view of a dental splint 1 according to the invention already arranged on a set of teeth. The dental splint 1 according to the invention has an upper jaw shell 2 and a lower jaw shell 3, the two shells 2, 3 being fixedly connected to each other via a connecting bar 4. It is advantageous if the connecting bar 4 is arranged on both sides exclusively in the area of the premolars and molars. The dental splint 1 is preferably designed as a one-piece part, which is preferably produced as a 3D printed part, as shown in FIG. 2. Due to the material used, preferably a nylon, the dental splint 1 is elastically deformable and adapts flexibly to the teeth, but is nevertheless stable. The dental splint 1 according to the invention forms saliva ducts 5 between the dental splint 1 and the chewing surface 6 of the teeth 18 when the dental splint 1 is arranged on the dentition, which is clearly shown in FIG. 4. The saliva duct 5, which is formed between the dental splint 1 and the tooth 18, ensures a permanent flushing under of saliva while wearing the dental splint 1 during sleep.

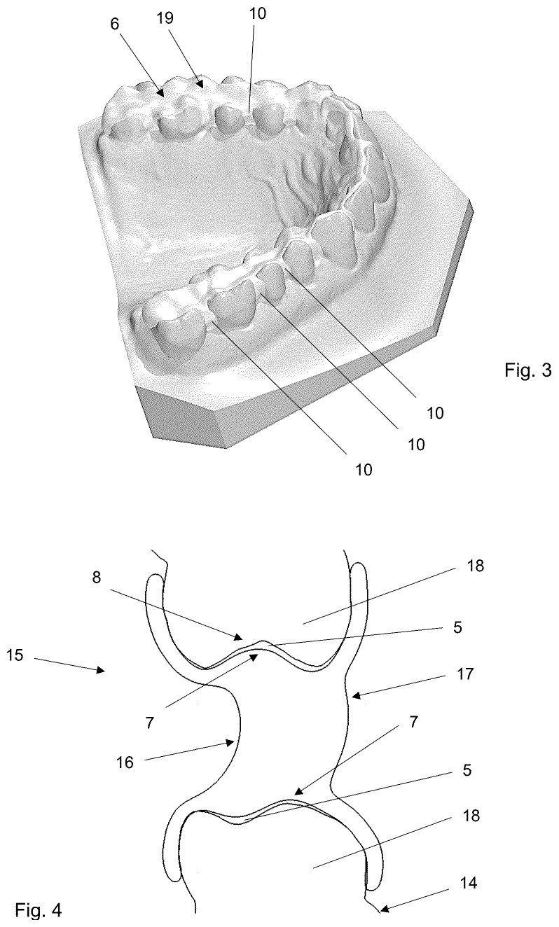

FIG. 3 shows the course of the saliva ducts 5 and the interdental passages 10 along which the saliva can flow. In the dental splint 1 according to the invention, this area, which is shown in FIG. 3 as a kind of filling mass, is hollow. FIG. 3 shows that the hollow saliva ducts 5 present in the dental splint 1 and the hollow interdental passages 10 are filled with a spacer material 19 to produce the dental splint 1, wherein this representation can be used as a representation of the course of the saliva ducts 5 and the interdental passages 10.

The saliva ducts 5 are formed by a saliva duct edge 7, which is arranged on the dental splint 1 and the chewing surface 6, as shown in FIG. 4. The saliva ducts 5 preferably run along the dental fissures 8 in the chewing surfaces 6. This is again clearly visible in FIG. 3, where the course of the saliva ducts 5 is shown by means of the spacer material 19. Even in the labial region where there are no chewing surfaces, the saliva duct 5 runs along the incisors and canines in the lingual and palatal region.

In order to increase the flexibility of the dental splint 1 on the one hand and, on the other hand, to ensure that saliva is flushed under the dental splint or around the teeth, the dental splint 1 preferably has interdental openings 9, which are positioned between the individual teeth 18, both directed towards the inside of the mouth and towards the outside. The interdental openings 9 connect to the interdental passages 10, which connect to the saliva ducts 5, which ensures optimum saliva flow and prevents bacteria from accumulating in stagnant pools of saliva.

In order to ensure that the dental splint 1 adapts to the teeth and is flexible, it is advantageous if the dental splint 1 has buccal and labial openings 11, 12, which are preferably aligned with the tooth surfaces. In addition, it is advantageous if the buccal and labial openings 11, 12 are arranged above or below the widest point of a respective tooth. In other words, the buccal and labial openings 11, 12 in the lower jaw shell 3 are above the widest point of the respective tooth 18 and in the upper jaw shell 2 below the widest point of the tooth 18, this allows the splint to fit snugly against the teeth 18 and still ensures a good hold of the dental splint 1 on the dentition due to the slight pretension generated by the points where no openings are provided. In most cases, the buccal and labial openings 11, 12 are larger than the interdental openings 9.

The tooth necks 14 are exposed on all teeth 18, the splint 1 ends above the tooth necks 14, which ensures a high level of wearing comfort and also supports the flow of saliva. To ensure proper breathing when wearing the dental splint 1, the dental splint has a breathing opening 13 in the labial area.

While specific embodiments of the invention have been shown and described in detail to illustrate the inventive principles, it will be understood that the invention may be embodied otherwise without departing from such principles.

I claim:

1. A dental splint for locking the lower jaw, preferably in a predefined protrusion relationship of the upper and lower jaw, comprising: an upper jaw shell; a lower jaw shell; and a connecting bar that connects the upper jaw shell to the lower jaw shell, wherein the dental splint is configured so that, when the dental splint is worn during sleep, saliva ducts are formed, in a region of chewing surfaces of teeth of a wearer, between the dental splint and the chewing surfaces so as to ensure permanent flushing of saliva under the dental splint while the dental splint is being worn during sleep.

2. The dental splint according to claim 1, wherein the dental splint is configured to lock the lower jaw in a predefined protrusion relationship of the lower jaw and and upper jaw.

3. The dental splint according to claim 1, further comprising a saliva duct edge, wherein the saliva ducts are formed between the saliva duct edge and the chewing surfaces of the teeth.

4. The dental splint according to claim 1, wherein the saliva ducts are arranged to run along fissures in the chewing surfaces of the teeth.

5. The dental splint according to claim 1, wherein the upper jaw shell, the lower jaw shell and the connecting bar form a single part.

6. The dental splint according to claim 1, comprising interdental openings, the interdental openings positioned so as to be between the teeth.

7. The dental splint according to claim 6, wherein the interdental openings and the saliva ducts arranged in the region of the chewing surfaces are connected to one another by interdental passages in the dental splint.

8. The dental splint according to claim 1, further comprising buccally arranged openings.

9. The dental splint according to claim 1, further comprising labially arranged openings.

10. The dental splint according to claim 1, wherein an end of the dental splint is configured so that tooth necks are exposed.

11. The dental splint according to claim 1, wherein a breathing opening is arranged in a labial region between the upper jaw shell and the lower jaw shell.

12. The dental splint according to claim 1, wherein the dental splint is a 3D printed part.

13. The dental splint according to claim 1, wherein the dental splint is made of a nylon material.

14. The dental splint according to claim 13, wherein the nylon material is a medically certified biocompatible nylon material.
